(19) United States
(12) Patent Application Publication
(10) Pub. No.: US 2007/0100181 A1
(43) Pub. Date: May 3, 2007

(54) Olefin isomerization

(76) Inventors: Mark Andrew Harmer, Kennett Square, PA (US); Christopher P. Junk, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/583,342

(22) Filed: Oct. 19, 2006

Related U.S. Application Data
(60) Provisional application No. 60/730,716, filed on Oct. 27, 2005.

(51) Int. Cl. C07C 5/22

(52) U.S. Cl. 585/253; 585/255

(57) ABSTRACT

This invention relates to a process for isomerizing olefins; the reaction is carried out in the presence of at least one ionic liquid.

### FIELD OF THE INVENTION

This invention relates to a process for isomerizing olefins; the reaction is carried out in the presence of at least one ionic liquid.

### BACKGROUND

The isomerization of olefins to internal olefins is an important reaction in the refining industry. Long chain olefins, for example, can be isomerized to the internal olefins, which can be used as precursors to materials used in lubrication.

Various methods for the catalytic isomerization of olefins have been disclosed. See, for example, Dunning, H. N. (Ind. Eng. Chem. (1953) 45:551-564) and U.S. Pat. No. 5,849,974. Homogeneous catalysts have the disadvantage that the product or products from the isomerization reaction require separation from the reaction catalyst. A cost effective, efficient process for the production and purification of olefin isomers is required.

Ionic liquids are liquids composed of ions that are liquid around or below 100° C. (Science (2003) 302:792-793). Ionic liquids exhibit negligible vapor pressure, and with increasing regulatory pressure to limit the use of traditional industrial solvents due to environmental considerations such as volatile emissions and aquifer and drinking water contamination, much research has been devoted to designing ionic liquids that could function as replacements for conventional solvents.

### SUMMARY OF THE INVENTION

The present invention provides a process for carrying out isomerization reactions using ionic liquids as solvent. The use of at least one ionic liquid as the solvent for this reaction allows for ready separation of the product(s) from the catalyst.

The present invention relates to a process for making internal olefins comprising:

(A) forming a reaction mixture comprising (1) at least one α-olefin having from 4 to 25 carbons, (2) at least one acid catalyst selected from the group consisting of rare earth fluorinated alkyl sulfonates, organic sulfonic acids, fluoroalkyl sulfonic acids, metal sulfonates, metal trifluoroacetates, and combinations thereof, and (3) at least one ionic liquid having the Formula Z⁺A⁻, wherein Z⁺ and A⁻ are defined in the Detailed Description;

thereby forming an isomer phase comprising at least one internal olefin and an ionic liquid phase that comprises the at least one acid catalyst; and

B) separating the isomer phase from the ionic liquid phase, thereby forming a separated ionic liquid phase.

### BRIEF DESCRIPTION OF THE FIGURES

FIG. 1 shows the reaction mixture produced from contacting 1-dodecene with 1,1,2,2-tetrafluoroethanesulfonic acid in the presence of an ionic liquid.

FIG. 2 shows the two-phase product obtained by contacting 1-dodecene with 1,1,2,2-tetrafluoroethanesulfonic acid.

FIG. 3 is a GC tracing of the isomer phase obtained from the isomerization of 1-dodecene in the presence of the catalyst 1,1,2,2-tetrafluoroethanesulfonic acid and the ionic liquid 1-dodecyl-3-methylimidazolium 1,1,2,2-tetrafluoroethanesulfonate.

FIG. 4 is a GC tracing of the reaction products obtained from the isomerization of 1-dodecene in the presence of the catalyst 1,1,2,2-tetrafluoroethanesulfonic acid (no ionic liquid present).

FIG. 5 is a GC tracing of the isomer phase obtained from the isomerization of 1-dodecene in the presence of the catalyst 1,1,2,2-tetrafluoroethanesulfonic acid and the ionic liquid 1-octadecyl-3-methylimidazolium 1,1,2,2-tetrafluoroethanesulfonate.

### DETAILED DESCRIPTION OF THE INVENTION

The present invention relates to a process for isomerizing α-olefins in the presence of an ionic liquid solvent. The use of an ionic liquid as the solvent for the isomerization reaction is advantageous because the acid catalyst is recovered in an ionic liquid phase that is separate from the phase comprising the product isomer(s), thus the product isomer(s) are easily separated from the acid catalyst.

Definitions

In this disclosure a number of terms and abbreviations are used. The following definitions are provided.

By “ionic liquid” is meant an organic salt that is liquid around or below 100° C.

By “fluoroalkyl” is meant an alkyl group wherein at least one member selected from the hydrogens has been replaced by fluorine. By “perfluoroalkyl” is meant an alkyl group wherein all of the hydrogens have been replaced by fluorines.

By “alkoxy” is meant a straight-chain or branched alkyl group bound via an oxygen atom. By “fluoroalkoxy” is meant an alkoxy group wherein at least one member selected from the hydrogens has been replaced by fluorine. By “perfluoroalkoxy” is meant an alkoxy group wherein all of the hydrogens have been replaced by fluorines.

By “halogen” is meant bromine, iodine, chlorine or fluorine.

By “heteroaryl” is meant an aryl group having one or more heteroatoms.

By “catalyst” is meant a substance that affects the rate of the reaction but not the reaction equilibrium, and emerges from the process chemically unchanged.

When referring to an alkane, alkene, alkoxy, fluoroalkoxy, perfluoroalkoxy, fluoroalkyl, perfluoroalkyl, aryl or heteroaryl, the term “optionally substituted with at least one member selected from the group consisting of” means that one or more hydrogens on the carbon chain may be independently substituted with one or more of at least one member of the group. For example, substituted C₂H₅ may be, without limitations, CF₂CF₃, CH₂CH₂OH or CF₂CF₂I.

The expression “C₁ to Cn straight-chain or branched”, where n is an integer defining the length of the carbon chain, is meant to indicate that C₁ and C₂ are straight-chain, and C₃ to Cₙ may be straight-chain or branched.

The invention provides a process for making internal olefins comprising:

(A) forming a reaction mixture comprising (1) at least one α-olefin having from 4 to 25 carbons, (2) at least one acid catalyst selected from the group consisting of rare earth fluorinated alkyl sulfonates, organic sulfonic acids, fluoroalkyl sulfonic acids, metal sulfonates, metal trifluoroacetates, and combinations thereof, and (3) at least one ionic liquid having the Formula Z⁺A⁻, wherein Z⁺ is a cation selected from the group consisting of: 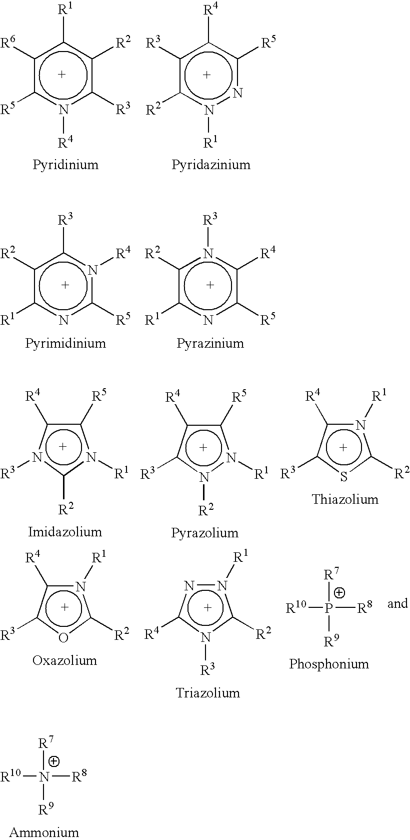
wherein R¹, R², R³, R⁴, R⁵and R⁶ are independently selected from the group consisting of:
(i) H
(ii) halogen
(iii) —CH₃, —C₂H₅, or C₃ to C₂₅, preferably C₄ to C₂₀, straight-chain, branched or cyclic alkane or alkene, optionally substituted with at least one member selected from the group consisting of Cl, Br, F, I, OH, NH₂ and SH;
(iv) —CH₃, —C₂H₅, or C₃ to C₂₅, preferably C₄ to C₂₀, straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
(v) C₆ to C₂₅ unsubstituted aryl or unsubstituted heteroaryl having one to three heteroatoms independently selected from the group consisting of O, N and S; and
(vi) C₆ to C₂₅ substituted aryl or substituted heteroaryl having one to three heteroatoms independently selected from the group consisting of O, N and S; and wherein said substituted aryl or substituted heteroaryl has one to three substituents independently selected from the group consisting of
   (1) —CH₃, —C₂H₅, or C₃ to C₂₅, preferably C₄ to C₂₀, straight-chain, branched or cyclic alkane or alkene, optionally substituted with at least one member selected from the group consisting of Cl, Br, F, I, OH, NH₂ and SH,
   (2) OH,
   (3) NH₂, and
   (4) SH;
      R⁷, R⁸, R⁹, and R¹⁰ are independently selected from the group consisting of:
(vii) —CH₃, —C₂H₅, or C₃ to C₂₅, preferably C₄ to C₂₀, straight-chain, branched or cyclic alkane or alkene, optionally substituted with at least one member selected from the group consisting of Cl, Br, F, I, OH, NH₂ and SH;
(viii) —CH₃, —C₂H₅, or C₃ to C₂₅, preferably C₄ to C₂₀, straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
(ix) C₆ to C₂₅ unsubstituted aryl, or C₃ to C₂₅ unsubstituted heteroaryl having one to three heteroatoms independently selected from the group consisting of O, N and S; and
(x) C₆ to C₂₅ substituted aryl, or C₃ to C₂₅ substituted heteroaryl having one to three heteroatoms independently selected from the group consisting of O, N and S; and wherein said substituted aryl or substituted heteroaryl has one to three substituents independently selected from the group consisting of
   (1) —CH₃, —C₂H₅, or C₃ to C₂₅, preferably C₄ to C₂₀, straight-chain, branched or cyclic alkane or alkene, optionally substituted with at least one member selected from the group consisting of Cl, Br, F, I, OH, NH₂ and SH,
   (2) OH,
   (3) NH₂, and
   (4) SH;
wherein optionally at least two of R¹, R², R³, R⁴, R⁵, R⁶, R⁷, R⁸, R⁹, and R¹⁰ can together form a cyclic or bicyclic alkanyl or alkenyl group; and
A⁻ is R¹¹—SO₃⁻ or (R¹²—SO₂)₂N⁻; wherein R¹¹ and R¹² are independently selected from the group consisting of:
   (a) —CH₃, —C₂H₅, or C₃ to C₂₅, preferably C₄ to C₂₀, straight-chain, branched or cyclic alkane or alkene, optionally substituted with at least one member selected from the group consisting of Cl, Br, F, I, OH, NH₂ and SH;
   (b) —CH₃, —C₂H₅, or C₃ to C₂₅, preferably C₄ to C₂₀, straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
   (c) C₆ to C₂₅ unsubstituted aryl or unsubstituted heteroaryl having one to three heteroatoms independently selected from the group consisting of O, N and S; and
   (d) C₆ to C₂₅ substituted aryl or substituted heteroaryl having one to three heteroatoms independently selected from the group consisting of O, N and S; and wherein said substituted aryl or substituted heteroaryl has one to three substituents independently selected from the group consisting of:
      (1) —CH₃, —C₂H₅, or C₃ to C₂₅, preferably C₄ to C₂₀, straight-chain, branched or cyclic alkane or alkene, optionally substituted with at least one member selected from the group consisting of Cl, Br, F, I, OH, NH₂ and SH,
      (2) OH,
      (3) NH₂, and
      (4) SH;

thereby forming an isomer phase comprising at least one internal olefin and an ionic liquid phase that comprises the at least one acid catalyst; and

B) separating the isomer phase from the ionic liquid phase, thereby forming a separated ionic liquid phase.

In a more specific embodiment, Z⁺ is imidazolium or phosphonium.

In another more specific embodiment, A⁻ is selected from the group consisting of: [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CF₂CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, [(CF₃CFHCF₂SO₂)₂N]⁻.

In an even more specific embodiment, the ionic liquid Z⁺A⁻ is selected from the group consisting of 1-butyl-2,3-dimethylimidazolium1,1,2,2-tetrafluoroethanesulfonate, 1-butyl-methylimidazolium1,1,2,2-tetrafluoroethanesulfonate, 1-ethyl-3-methylimidazolium1,1,2,2-tetrafluoroethanesulfonate, 1-ethyl-3-methylimidazolium1,1,2,3,3,3-hexafluoropropanesulfonate, 1-hexyl-3-methylimidazolium1,1,2,2-tetrafluoroethanesulfonate, 1-dodecyl-3-methylimidazolium1,1,2,2-tetrafluoroethanesulfonate, 1-hexadecyl-3-methylimidazolium1,1,2,2-tetrafluoroethanesulfonate, 1-octadecyl-3-methylimidazolium1,1,2,2-tetrafluoroethanesulfonate, 1-propyl-3-(1,1,2,2-tetrafluoroethyl)imidazolium1,1,2,2-tetrafluoroethanesulfonate, 1-(1,1,2,2-tetrafluoroethyl)-3-(3,3,4,4,5,5,6,6,7,7,8,8,8-tridecafluorooctyl)imidazolium1,1,2,2-tetrafluoroethanesulfonate, 1-butyl-3-methylimidazolium1,1,2,3,3,3-hexafluoropropanesulfonate, 1-butyl-3-methylimidazolium1,1,2-trifluoro-2-(trifluoromethoxy)ethanesulfonate, 1-butyl-3-methylimidazolium1,1,2-trifluoro-2-(perfluoroethoxy)ethanesulfonate, 1-butyl-3-methylimidazolium1,1,2-trifluoro-2-(perfluoropropoxy)ethanesulfonate, tetradecyl(tri-n-hexyl)phosphonium1,1,2-trifluoro-2-(perfluoroethoxy)ethanesulfonate, tetradecyl(tri-n-butyl)phosphonium1,1,2,3,3,3-hexafluoropropanesulfonate, tetradecyl(tri-n-hexyl)phosphonium1,1,2-trifluoro-2-(trifluoromethoxy)ethanesulfonate, 1-ethyl-3-methylimidazolium 1,1,2,2-tetrafluoro-2-(pentafluoroethoxy)sulfonate, 1-ethyl-3-methylimidazolium1,1,2,2-tetrafluoro-2-(perfluoropropoxy)sulfonate, (3,3,4,4,5,5,6,6,7,7,8,8,8-tridecafluorooctyl)-trioctylphosphonium1,1,2,2-tetrafluoroethanesulfonate, 1-methyl-3-(3,3,4,4,5,5,6,6,7,7,8,8,8-tridecafluorooctyl)imidazolium1,1,2,2-tetrafluoroethanesulfonate, tetra-n-butylphosphonium 1,1,2-trifluoro-2-(trifluoromethoxy)ethanesulfonate, tetra-n-butylphosphonium1,1,2-trifluoro-2-(perfluoroethoxy)ethanesulfonate and tetra-n-butylphosphonium1,1,2-trifluoro-2-(perfluoropropoxy)ethanesulfonate.

The α-olefin starting material comprises from about four carbons to about twenty-five carbons. In a more specific embodiment, the α-olefin starting material may comprise from about 12 carbons to about 18 carbons. The starting material may comprise either linear or branched olefins, however preferably the starting material will comprise greater than 60 mol % linear α-olefin. The starting material may also comprise from about 10 mol % to about 35 mol % branched α-olefin, from about 0 mol % to about 10 mol % linear internal olefin, and/or from about 0 mol % to about 10 mol % branched internal olefin. The olefin starting material may also be admixed with one or more inert hydrocarbons, such as paraffins, cycloparaffins, or aromatics, however preferably, the olefin starting material comprises at least 90% by weight of olefins.

The at least one acid catalyst usable in the current process is selected from the group consisting of rare earth fluorinated alkyl sulfonates, organic sulfonic acids, fluoroalkyl sulfonic acids, metal sulfonates, metal trifluoroacetates, and combinations thereof.

In a preferred embodiment, the at least one acid catalyst is selected from the group consisting of:

(i) bismuth triflate;

(ii) yttrium triflate;

(iii) ytterbium triflate;

(iv) neodymium triflate;

(v) lanthanum triflate;

(vi) scandium triflate;

(vii) zirconium triflate;

(viii) Formula (I); 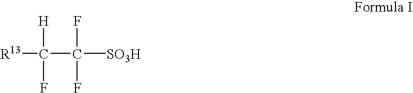
wherein:
R¹³ is selected from the group consisting of:
(1) halogen;
(2) —CH₃, —C₂H₅ or C₃ to C₁5, preferably C₃ to C₆, straight-chain or branched alkane or alkene, optionally substituted with at least one member selected from the group consisting of Cl, Br, I, OH, NH₂ and SH;
(3) —OCH₃, —OC₂H₅ or C₃ to C₁₅, preferably C₃ to C₆, straight-chain or branched alkoxy, optionally substituted with at least one member selected from the group consisting of Cl, Br, I, OH, NH₂ and SH;
(4) C₁ to C₁₅, preferably C₃ to C₆, straight-chain or branched fluoroalkyl, optionally substituted with at least one member selected from the group consisting of Cl, Br, I, OH, NH₂ and SH;
(5) C₁ to C₁₅, preferably C₃ to C₆, straight-chain or branched fluoroalkoxy, optionally substituted with at least one member selected from the group consisting of Cl, Br, I, OH, NH₂ and SH;
(6) C₁ to C₁₅, preferably C₃ to C₆, straight-chain or branched perfluoroalkyl; and
(7) C₁ to C₁₅, preferably C₃ to C₆, straight-chain or branched perfluoroalkoxy;
(ix) Formula (II) 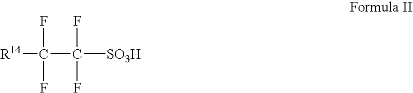
   wherein:
   R¹⁴ is selected from the group consisting of:
   (1) —CH₃, —C₂H₅ or C₃ to C₁₅, preferably C₃ to C₆, straight-chain or branched alkoxy, optionally substituted with at least one member selected from the group consisting of Cl, Br, I, OH, NH₂ and SH;
   (2) C₁ to C₁₅, preferably C₃ to C₆, straight-chain or branched fluoroalkoxy, optionally substituted with at least one member selected from the group consisting of Cl, Br, I, OH, NH₂ and SH; and
   (3) C₁ to C₁₅, preferably C₃ to C₆, straight-chain or branched perfluoroalkoxy; and
(x) Formula (III); 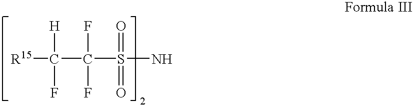
   wherein:
   R¹⁵ is selected from the group consisting of:
   (1) halogen;
   (2) —CH₃, —C₂H₅ or C₃ to C₁₅, preferably C₃ to C₆, straight-chain or branched alkane or alkene, optionally substituted with at least one member selected from the group consisting of Cl, Br, I, OH, NH₂ and SH;
   (3) —OCH₃, —OC₂H₅ or C₃ to C₁₅, preferably C₃ to C₆, straight-chain or branched alkoxy, optionally substituted with at least one member selected from the group consisting of Cl, Br, I, OH, NH₂ and SH;
   (4) C₁ to C₁₅, preferably C₃ to C₆, straight-chain or branched fluoroalkyl, optionally substituted with at least one member selected from the group consisting of Cl, Br, I, OH, NH₂ and SH;
   (5) C₁ to C₅, preferably C₃ to C₆, straight-chain or branched fluoroalkoxy, optionally substituted with at least one member selected from the group consisting of Cl, Br, I, OH, NH₂ and SH;
   (6) C₁ to C₁₅, preferably C₃ to C₆, straight-chain or branched perfluoroalkyl; and
   (7) C₁ to C₁₅, preferably C₃ to C6, straight-chain or branched perfluoroalkoxy.

In a more specific embodiment, the at least one acid catalyst is 1,1,2,2-tetrafluoroethanesulfonic acid, 1,1,2,3,3,3-hexafluoropropanesulfonic acid, 2-chloro-1,1,2-trifluoroethanesulfonic acid, 1,1,2-trifluoro-2-(perfluoroethoxy)ethanesulfonic acid, 1,1,2-trifluoro-2-(trifluoromethoxy)ethanesulfonic acid, or 1,1,2-trifluoro-2-(perfluoropropoxy)ethanesulfonic acid.

Most of the catalysts may be obtained commercially. The catalysts not available commercially may be synthesized as described in the following references: U.S. Pat. No. 2,403,207, Rice, et al. (Inorg. Chem., 1991, 30:4635-4638), Coffman, et al. (J. Org. Chem., 1949, 14:747-753 and Koshar, et al. (J. Am. Chem. Soc. (1953) 75:4595-4596).

The at least one acid catalyst is used at a concentration of from about 0.1% to about 20% by weight of the total weight of the α-olefin(s) at the start of the reaction. In a more specific embodiment, the at least one acid catalyst is used at a concentration of from about 0.1% to about 10% by weight of the total weight of the α-olefin(s) at the start of the reaction. In an even more specific embodiment, the at least one acid catalyst is used at a concentration of from about 0.1% to about 5% by weight of the total weight of the α-olefin(s) at the start of the reaction.

The reaction is preferably carried out at a temperature of from about 50° C. to about 175° C. In a more specific embodiment, the reaction is carried out at a temperature of from about 50° C. to about 120° C.

The reaction is preferably carried out under an inert atmosphere, such as nitrogen, argon or helium. The reaction may be performed at atmospheric pressure, or at pressures above atmospheric pressure.

The time for the reaction will depend on many factors, such as the reactants, reaction conditions and reactor. One skilled in the art will know to adjust the time for the reaction to achieve optimal isomerization of the α-olefins.

Cations and Anions of the Ionic Liquids

Cations of ionic liquids useful for the invention are available commercially, or may be synthesized by methods known to those skilled in the art. The fluoroalkyl sulfonate anions may be synthesized from perfluorinated terminal olefins or perfluorinated vinyl ethers generally according to the method of Koshar, et al. (J. Am. Chem. Soc. (1953) 75:4595-4596); in one embodiment, sulfite and bisulfite are used as the buffer in place of bisulfite and borax, and in another embodiment, the reaction is carried in the absence of a radical initiator. 1,1,2,2-Tetrafluoroethanesulfonate, 1,1,2,3,3,3-hexafluoropropanesulfonate, 1,1,2-trifluoro-2-(trifluoromethoxy)ethanesulfonate, and 1,1,2-trifluoro-2-(pentafluoroethoxy)ethanesulfonate may be synthesized according to Koshar, et al. (supra), with modifications. Preferred modifications include using a mixture of sulfite and bisulfite as the buffer, freeze drying or spray drying to isolate the crude 1,1,2,2-tetrafluoroethanesulfonate and 1,1,2,3,3,3-hexafluoropropanesulfonate products from the aqueous 5 reaction mixture, using acetone to extract the crude 1,1,2,2-tetrafluoroethanesulfonate and 1,1,2,3,3,3-hexafluoropropanesulfonate salts, and crystallizing 1,1,2-trifluoro-2-(trifluoromethoxy)ethanesulfonate and 1,1,2-trifluoro-2-(pentafluoroethoxy)ethanesulfonate from the reaction mixture by cooling.

The at least one ionic liquid useful for the invention may be obtained commercially, or may be synthesized using the cations and anions by methods well known to those skilled in the art.

General Procedure for Synthesizing Ionic Liquids that are not Miscible with Water:

Solution #1 is made by dissolving a known amount of the halide salt of the cation in deionized water. This may involve heating to ensure total dissolution. Solution #2 is made by dissolving an approximately equimolar amount (relative to the cation) of the potassium or sodium salt of the anion in deionized water. This may also involve heating to ensure total dissolution. Although it is not necessary to use equimolar quantities of the cation and anion, a 1:1 equimolar ratio minimizes the impurities obtained by the reaction. The two aqueous solutions (#1 and #2) are mixed and stirred at a temperature that optimizes the separation of the desired product phase as either an oil or a solid on the bottom of the flask. In one embodiment, the aqueous solutions are mixed and stirred at room temperature, however the optimal temperature may be higher or lower based on the conditions necessary to achieve optimal product separation. The water layer is separated, and the product is washed several times with deionized water to remove chloride or bromide impurities. An additional base wash may help to remove acidic impurities. The product is then diluted with an appropriate organic solvent (chloroform, methylene chloride, etc.) and dried over anhydrous magnesium sulfate or other preferred drying agent. The appropriate organic solvent is one that is miscible with the ionic liquid and that can be dried. The drying agent is removed by suction filtration and the organic solvent is removed in vacuo. High vacuum is applied for several hours or until residual water is removed. The final product is usually in the form of a liquid, and in any case are liquid around or below 100° C.

General Procedure for the Synthesis of Ionic Liquids that are Miscible with Water:

Solution #1 is made by dissolving a known amount of the halide salt of the cation in an appropriate solvent. This may involve heating to ensure total dissolution. Preferably the solvent is one in which the cation and anion are miscible, and in which the salts formed by the reaction are minimally miscible; in addition, the appropriate solvent is preferably one that has a relatively low boiling point such that the solvent can be easily removed after the reaction. Appropriate solvents include, but are not limited to, high purity dry acetone, alcohols such as methanol and ethanol, and acetonitrile. Solution #2 is made by dissolving an equimolar amount (relative to the cation) of the salt (generally potassium or sodium) of the anion in an appropriate solvent, typically the same as that used for the cation. This may also involve heating to ensure total dissolution. The two solutions (#1 and #2) are mixed and stirred under conditions that result in approximately complete precipitation of the halide salt byproduct (generally potassium halide or sodium halide); in one embodiment of the invention, the solutions are mixed and stirred at approximately room temperature for about 4-12 hours. The halide salt is removed by suction filtration through an acetone/celite pad, and color can be reduced through the use of decolorizing carbon as is known to those skilled in the art. The solvent is removed in vacuo and then high vacuum is applied for several hours or until residual water is removed. The final product is usually in the form of a liquid, and in any case are liquid around or below 100° C.

The physical and chemical properties of ionic liquids can be specifically selected by choice of the appropriate cation and anion. For example, increasing the chain length of one or more alkyl chains of the cation will affect properties such as the melting point, hydrophilicity/lipophilicity, density and solvation strength of the ionic liquid. Choice of the anion can affect, for example, the melting point, the water solubility and the acidity and coordination properties of the composition. Effects of cation and anion on the physical and chemical properties of ionic liquids are known to those skilled in the art and are reviewed in detail by Wasserscheid and Keim (Angew. Chem. Int. Ed. (2000) 39:3772-3789) and Sheldon (Chem. Commun. (2001) 2399-2407). In the present invention, the choice of the ionic liquid may affect the degree of formation of internal olefins. In addition, as shown in Examples 1 and 2, the ionic liquid can increase the activity of the catalyst.

The process of the present invention may be carried out in batch, sequential batch (i.e., a series of batch reactors) or in continuous mode in any of the equipment customarily employed for continuous process (see for example, H. S. Fogler, Elementary Chemical Reaction Engineering, Prentice-Hall, Inc., N.J., USA).

An advantage to the use of at least one ionic liquid in this reaction is that the reaction product comprises an isomer phase comprising the internal olefin(s) and an ionic liquid phase that comprises the acid catalyst. Thus the internal olefin(s) is/are easily recoverable from the acid catalyst by, for example, decantation. In a preferred embodiment, the separated ionic liquid phase is reused to form the reaction mixture.

### EXAMPLES

General Materials and Methods

The following abbreviations are used:

Nuclear magnetic resonance is abbreviated NMR; gas chromatography is abbreviated GC; gas chromatography-mass spectrometry is abbreviated GC-MS; thin layer chromatography is abbreviated TLC; thermogravimetric analysis (using a Universal V3.9A TA instrument analyzer (TA Instruments, Inc., Newcastle, Del.)) is abbreviated TGA. Centigrade is abbreviated C, megapascal is abbreviated MPa, gram is abbreviated g, kilogram is abbreviated kg, milliliter(s) is abbreviated mL(s), hour is abbreviated hr; weight percent is abbreviated wt %; milliequivalents is abbreviated meq; melting point is abbreviated Mp; differential scanning calorimetry is abbreviated DSC.

1-Butyl-2,3-dimethylimidazolium chloride, 1-hexyl-3-methylimidazolium chloride, 1-dodecyl-3-methylimidazolium chloride, 1-hexadecyl-3-methyl imidazolium chloride, 1-octadecyl-3-methylimidazolium chloride, imidazole, tetrahydrofuran, iodopropane, acetonitrile, iodoperfluorohexane, toluene, 1,3-propanediol, oleum (20% SO₃), sodium sulfite (Na₂SO₃, 98%), and acetone were obtained from Acros (Hampton, N.H.). Potassium metabisulfite (K₂S₂O₅, 99%), was obtained from Mallinckrodt Laboratory Chemicals (Phillipsburg, N.J.). Potassium sulfite hydrate (KHSO₃•xH₂O, 95%), sodium bisulfite (NaHSO₃), sodium carbonate, magnesium sulfate, ethyl ether, 1,1,1,2,2,3,3,4,4,5,5,6,6-tridecafluoro-8-iodooctane, trioctyl phosphine, 1-dodecene, and 1-ethyl-3-methylimidazolium chloride (98%) were obtained from Aldrich (St. Louis, Mo.). Sulfuric acid and methylene chloride were obtained from EMD Chemicals, Inc. (Gibbstown, N.J.). Perfluoro(ethyl vinyl ether), perfluoro(methyl vinyl ether), hexafluoropropene and tetrafluoroethylene were obtained from DuPont Fluoroproducts (Wilmington, Del.). 1-Butyl-methylimidazolium chloride was obtained from Fluka (Sigma-Aldrich, St. Louis, Mo.). Tetra-n-butylphosphonium bromide and tetradecyl(tri-n-hexyl)phosphonium chloride were obtained from Cytec (Canada Inc., Niagara Falls, Ontario, Canada). 1,1,2,2-Tetrafluoro-2-(pentafluoroethoxy)sulfonate was obtained from SynQuest Laboratories, Inc. (Alachua, Fla.).

Preparation of Anions not Generally Available Commercially

### (A) Synthesis of potassium 1.1,2,2-tetrafluoroethanesulfonate (TFES-K):

A 1-gallon Hastelloy® C276 reaction vessel was charged with a solution of potassium sulfite hydrate (176 g, 1.0 mol), potassium metabisulfite (610 g, 2.8 mol) and deionized water (2000 mL). The pH of this solution was 5.8. The vessel was cooled to 18° C., evacuated to 0.10 MPa, and purged with nitrogen. The evacuate/purge cycle was repeated two more times. To the vessel was then added tetrafluoroethylene (TFE, 66 g), and it was heated to 100° C. at which time the inside pressure was 1.14 MPa. The reaction temperature was increased to 125° C. and kept there for 3 hr. As the TFE pressure decreased due to the reaction, more TFE was added in small aliquots (20-30 g each) to maintain operating pressure roughly between 1.14 and 1.48 MPa. Once 500 g (5.0 mol) of TFE had been fed after the initial 66 g precharge, the vessel was vented and cooled to 25° C. The pH of the clear light yellow reaction solution was 10-11. This solution was buffered to pH 7 through the addition of potassium metabisulfite (16 g).

The water was removed in vacuo on a rotary evaporator to produce a wet solid. The solid was then placed in a freeze dryer (Virtis Freezemobile 35xl; Gardiner, N.Y.) for 72 hr to reduce the water content to approximately 1.5 wt % (1387 g crude material). The theoretical mass of total solids was 1351 g. The mass balance was very close to ideal and the isolated solid had slightly higher mass due to moisture. This added freeze drying step had the advantage of producing a free-flowing white powder whereas treatment in a vacuum oven resulted in a soapy solid cake that was very difficult to remove and had to be chipped and broken out of the flask.

The crude TFES-K can be further purified and isolated by extraction with reagent grade acetone, filtration, and drying.

¹⁹F NMR (D₂O) δ −122.0 (dt, J_{FH}=6 Hz, J_{FF}=6 Hz, 2F); −136.1 (dt, J_{FH}=53 Hz, 2F). ¹H NMR (D₂O) δ 6.4 (tt, J_{FH}=53 Hz, J_{FH}=6 Hz, 1H). % Water by Karl-Fisher titration: 580 ppm. Analytical calculation for C₂HO₃F₄SK: C, 10.9: H, 0.5: N, 0.0 Experimental results: C, 11.1: H, 0.7: N, 0.2. Mp (DSC): 242° C. TGA (air): 10% wt. loss @ 367° C., 50% wt. loss @ 375° C. TGA (N₂): 10% wt. loss @ 363° C., 50% wt. loss @ 375° C.

### (B) Synthesis of potassium-1,1,2-trifluoro-2-(perfluoroethoxy)ethanesulfonate (TPES-K):

A 1-gallon Hastelloy® C276 reaction vessel was charged with a solution of potassium sulfite hydrate (88 g, 0.56 mol), potassium metabisulfite (340 g, 1.53 mol) and deionized water (2000 mL). The vessel was cooled to 7° C., evacuated to 0.05 MPa, and purged with nitrogen. The evacuate/purge cycle was repeated two more times. To the vessel was then added perfluoro(ethyl vinyl ether) (PEVE, 600 g, 2.78 mol), and it was heated to 125° C. at which time the inside pressure was 2.31 MPa. The reaction temperature was maintained at 125° C. for 10 hr. The pressure dropped to 0.26 MPa at which point the vessel was vented and cooled to 25° C. The crude reaction product was a white crystalline precipitate with a colorless aqueous layer (pH=7) above it.

The ¹⁹F NMR spectrum of the white solid showed pure desired product, while the spectrum of the aqueous layer showed a small but detectable amount of a fluorinated impurity. The desired product is less soluble in water so it precipitated in pure form.

The product slurry was suction filtered through a fritted glass funnel, and the wet cake was dried in a vacuum oven (60° C., 0.01 MPa) for 48 hr. The product was obtained as off-white crystals (904 g, 97% yield).

¹⁹F NMR (D₂O) δ −86.5 (s, 3F); −89.2, −91.3 (subsplit ABq, J_{FF}=147 Hz, 2F); −119.3, −121.2 (subsplit ABq, J_{FF}=258 Hz, 2F); −144.3 (dm, J_{FH}=53 Hz, 1F). ¹H NMR (D₂O) δ 6.7 (dm, J_{FH}=53 Hz, 1H). Mp (DSC) 263° C. Analytical calculation for C₄HO₄F₈SK: C, 14.3: H, 0.3 Experimental results: C, 14.1: H, 0.3. TGA (air): 10% wt. loss @ 359° C., 50% wt. loss @ 367° C. TGA (N₂): 10% wt. loss @ 362° C., 50% wt. loss @ 374° C.

### (C) Synthesis of potassium-1,1,2-trifluoro-2-(trifluoromethoxy)ethanesulfonate (TTES-K)

A 1-gallon Hastelloy® C276 reaction vessel was charged with a solution of potassium sulfite hydrate (114 g, 0.72 mol), potassium metabisulfite (440 g, 1.98 mol) and deionized water (2000 mL). The pH of this solution was 5.8. The vessel was cooled to −35° C., evacuated to 0.08 MPa, and purged with nitrogen. The evacuate/purge cycle was repeated two more times. To the vessel was then added perfluoro(methyl vinyl ether) (PMVE, 600 g, 3.61 mol) and it was heated to 125° C. at which time the inside pressure was 3.29 MPa. The reaction temperature was maintained at 125° C. for 6 hr. The pressure dropped to 0.27 MPa at which point the vessel was vented and cooled to 25° C. Once cooled, a white crystalline precipitate of the desired product formed leaving a colorless clear aqueous solution above it (pH=7).

The ¹⁹F NMR spectrum of the white solid showed pure desired product, while the spectrum of the aqueous layer showed a small but detectable amount of a fluorinated impurity.

The solution was suction filtered through a fritted glass funnel for 6 hr to remove most of the water. The wet cake was then dried in a vacuum oven at 0.01 MPa and 50° C. for 48 hr. This gave 854 g (83% yield) of a white powder. The final product was pure (by ¹⁹F and ¹H NMR) since the undesired byproduct remained in the water during filtration.

¹⁹F NMR (D₂O) δ −59.9 (d, J_{FH}=4 Hz, 3F); −119.6, −120.2 (subsplit ABq, J=260 Hz, 2F); −144.9 (dm, J_{FH}=53 Hz, 1F). ¹H NMR (D₂O) δ 6.6 (dm, J_{FH}=53 Hz, 1H). % Water by Karl-Fisher titration: 71 ppm. Analytical calculation for C₃HF₆SO₄K: C, 12.6: H, 0.4: N, 0.0 Experimental results: C, 12.6: H, 0.0: N, 0.1. Mp (DSC) 257° C. TGA (air): 10% wt. loss @ 343° C., 50% wt. loss @ 358° C. TGA (N₂): 10% wt. loss @ 341° C., 50% wt. loss @ 357° C.

### (D) Synthesis of sodium 1,1,2,3,3,3-hexafluoropropanesulfonate (HFPS-Na)

A 1-gallon Hastelloy® C reaction vessel was charged with a solution of anhydrous sodium sulfite (25 g, 0.20 mol), sodium bisulfite 73 g, (0.70 mol) and of deionized water (400 mL). The pH of this solution was 5.7. The vessel was cooled to 4° C., evacuated to 0.08 MPa, and then charged with hexafluoropropene (HFP, 120 g, 0.8 mol. 0.43 MPa). The vessel was heated with agitation to 120° C. and kept there for 3 hr. The pressure rose to a maximum of 1.83 MPa and then dropped down to 0.27 MPa within 30 minutes. At the end, the vessel was cooled and the remaining HFP was vented, and the reactor was purged with nitrogen. The final solution had a pH of 7.3.

The water was removed in vacuo on a rotary evaporator to produce a wet solid. The solid was then placed in a vacuum oven (0.02 MPa, 140° C., 48 hr) to produce 219 g of white solid which contained approximately 1 wt % water. The theoretical mass of total solids was 217 g.

The crude HFPS-Na can be further purified and isolated by extraction with reagent grade acetone, filtration, and drying.

¹⁹F NMR (D₂O) δ −74.5 (m, 3F); −113.1, −120.4 (ABq, J=264 Hz, 2F); −211.6 (dm, 1F). ¹H NMR (D₂O) δ 5.8 (dm, J_{FH}=43 Hz, 1H). Mp (DSC) 126° C. TGA (air): 10% wt. loss @ 326° C., 50% wt. loss @ 446° C. TGA (N₂): 10% wt. loss @ 322° C., 50% wt. loss @ 449° C.

Preparation of Catalysts not Generally Available Commercially

### (E) Synthesis of 1,1,2,2-tetrafluoroethanesulfonic acid (TFESA)

A 100 mL round bottomed flask with a sidearm and equipped with a digital thermometer and magnetic stirr bar was placed in an ice bath under positive nitrogen pressure. To the flask was added 50 g crude TFES-K (from synthesis (A) above), 30 g of concentrated sulfuric acid (95-98%) and 78 g oleum (20 wt % SO₃) while stirring. The amount of oleum was chosen such that there would be a slight excess of SO₃ after the SO₃ reacted with and removed the water in the sulfuric acid and the crude TFES-K. The mixing caused a small exotherm, which was controlled by the ice bath. Once the exotherm was over, a distillation head with a water condenser was placed on the flask, and the flask was heated under nitrogen behind a safety shield. The pressure was slowly reduced using a PTFE membrane vacuum pump (Buchi V-500, Buchi Analytical, Inc., Wilmington, Del.) in steps of 100 Torr (13 kPa) in order to avoid foaming. A dry-ice trap was placed between the distillation apparatus and the pump to collect any excess SO₃. When the pot temperature reached 120 degrees C. and the pressure was held at 20-30 Torr (2.7-4.0 kPa) a colorless liquid started to reflux which distilled at 110 degrees C. and 31 Torr (4.1 kPa). A forerun of lower-boiling impurity (2.0 g) was obtained before collecting 28 g of the desired colorless acid, TFESA.

It was calculated that approximately 39.8 g TFES-K was present in the 50 g of impure TFES-K. Thus, the 28 g of product is an 85% yield of TFESA from TFES-K, as well as an 85% overall yield from TFE. Analysis gave the following results: ¹⁹F NMR (CD₃OD) −125.2 dt, 3J_{FH}=6 Hz, 3J_{FF}=8 Hz, 2F); −137.6 (dt, 2J_{FH}=53 Hz, 2F). ¹H NMR (CD₃OD). 6.3 (tt, 3J_{FH}=6 Hz, 2J_{FH}=53 Hz, 1H).

### (F) Synthesis of 1,1,2,3,3,3-hexfluoropropanesulfonic acid (HFPSA)

A 100 mL round bottomed flask with a sidearm and equipped with a digital thermometer and magnetic stirr bar was placed in an ice bath under positive nitrogen pressure. To the flask was added 50 g crude sodium hexafluoropropanesulfonate (HFPS-Na) (from synthesis (D) above), 30 g of concentrated sulfuric acid (95-98%) and 58.5 g oleum (20 wt % SO₃) while stirring.

The amount of oleum was chosen such that there would be a slight excess of SO₃ after the SO₃ reacted with and removed the water in the sulfuric acid and the crude HFPSA. The mixing caused a small exotherm, which was controlled by the ice bath. Once the exotherm was over, a distillation head with a water condenser was placed on the flask, and the flask was heated under nitrogen behind a safety shield. The pressure was slowly reduced using a PTFE membrane vacuum pump in steps of 100 Torr (13 kPa) in order to avoid foaming. A dry-ice trap was placed between the distillation apparatus and the pump to collect any excess SO₃. When the pot temperature reached 100degrees C. and the pressure was held at 20-30 Torr (2.7-4 kPa) a colorless liquid started to reflux and later distilled at 118degrees C. and 23 Torr (3.1 kPa). A forerun of lower-boiling impurity (1.5 g) was obtained before collecting 36.0 g of the desired acid, hexafluoropropanesulfonic acid (HFPS).

It was calculated that approximately 44 g HFPS-Na was present in 50 g of impure HFPS-Na. Thus, the 36.0 g of HFPSA product was an 89% yield from HFPS-Na, as well as an 84% overall yield from HFP.

¹⁹F NMR (D₂O) −74.5 m, 3F); −113.1, −120.4 (ABq, J=264 Hz, 2F); −211.6 (dm, 1F). ¹H NMR (D₂O) 5.8 (dm, 2J_{FH}=43 Hz, 1H).

Preparation of Ionic Liquids

### (G) Synthesis of 1-butyl-2,3-dimethylimidazolium1,1,2,2-tetrafluoroethanesulfonate

1-Butyl-2,3-dimethylimidazolium chloride (22.8 g, 0.121 moles) was mixed with reagent-grade acetone (250 mL) in a large round-bottomed flask and stirred vigorously. Potassium 1,1,2,2-tetrafluoroethanesulfonate (TFES-K, 26.6 g, 0.121 moles) was added to reagent grade acetone (250 mL) in a separate round-bottomed flask, and this solution was carefully added to the 1-butyl-2,3-dimethylimidazolium chloride solution. The large flask was lowered into an oil bath and heated at 60° C. under reflux for 10 hours. The reaction mixture was then filtered using a large frit glass funnel to remove the white KCl precipitate formed, and the filtrate was placed on a rotary evaporator for 4 hours to remove the acetone. The product was isolated and dried under vacuum at 150° C. for 2 days.

¹H NMR (DMSO-d₆): δ 0.9 (t, 3H); 1.3 (m, 2H); 1.7 (m, 2H); 2.6 (s, 3H); 3.8 (s, 3H); 4.1 (t, 2H); 6.4 (tt, 1H); 7.58 (s, 1H); 7.62 (s, 1H). % Water by Karl-Fischer titration: 0.06%. TGA (air): 10% wt. loss @ 375° C., 50% wt. loss @ 415° C. TGA (N₂): 10% wt. loss @ 395° C., 50% wt. loss @ 425° C. The reaction scheme is shown below:

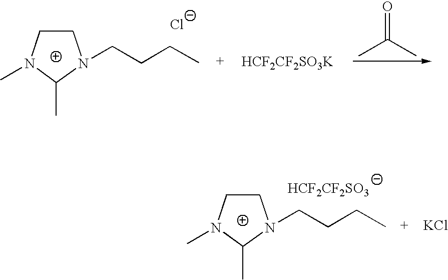

### (H) Synthesis of 1-butyl-3-methylimidazolium1,1,2,2-tetrafluoroethanesulfonate (Bmim-TFES)

1-Butyl-3-methylimidazolium chloride (60.0 g) and high purity dry acetone (>99.5%, 300 mL) were combined in a 1 liter flask and warmed to reflux with magnetic stirring until the solid completely dissolved. At room temperature in a separate 1 liter flask, potassium-1,1,2,2-tetrafluoroethanesulfonte (TFES-K, 75.6 g) was dissolved in high purity dry acetone (500 mL). These two solutions were combined at room temperature and allowed to stir magnetically for 2 hr under positive nitrogen pressure. The stirring was stopped and the KCl precipitate was allowed to settle, then removed by suction filtration through a fritted glass funnel with a celite pad. The acetone was removed in vacuo to give a yellow oil. The oil was further purified by diluting with high purity acetone (100 mL) and stirring with decolorizing carbon (5 g). The mixture was again suction filtered and the acetone removed in vacuo to give a colorless oil. This was further dried at 4 Pa and 25° C. for 6 hr to provide 83.6 g of product.

¹⁹F NMR (DMSO-d₆) δ −124.7 (dt, J=6 Hz, J=8 Hz, 2F); −136.8 (dt, J=53 Hz, 2F). ¹H NMR (DMSO-d₆) δ 0.9 (t, J=7.4 Hz, 3H); 1.3 (m, 2H); 1.8 (m, 2H); 3.9 (s, 3H); 4.2 (t, J=7 Hz, 2H); 6.3 (dt, J=53 Hz, J=6 Hz, 1H); 7.4 (s, 1H); 7.5 (s, 1H); 8.7 (s, 1H). % Water by Karl-Fisher titration: 0.14%. Analytical calculation for C₉H₁₂F₆N₂O₃S: C, 37.6: H, 4.7: N, 8.8. Experimental Results: C, 37.6: H, 4.6: N, 8.7. TGA (air): 10% wt. loss @ 380° C., 50% wt. loss @ 420° C. TGA (N₂): 10% wt. loss @ 375° C., 50% wt. loss @ 422° C.

### (I) Synthesis of 1-ethyl-3-methylimidazolium1,1,2,2-tetrafluoroethanesulfonate (Emim-TFES)

To a 500 mL round bottom flask was added 1-ethyl-3-methylimidazolium chloride (Emim-Cl, 98%, 61.0 g) and reagent grade acetone (500 mL). The mixture was gently warmed (50° C.) until almost all of the Emim-Cl dissolved. To a separate 500 mL flask was added potassium 1,1,2,2-tetrafluoroethanesulfonate (TFES-K, 90.2 g) along with reagent grade acetone (350 mL). This second mixture was stirred magnetically at 24° C. until all of the TFES-K dissolved.

These solutions were combined in a 1 liter flask producing a milky white suspension. The mixture was stirred at 24° C. for 24 hrs. The KCl precipitate was then allowed to settle leaving a clear green solution above it.

The reaction mixture was filtered once through a celite/acetone pad and again through a fritted glass funnel to remove the KCl. The acetone was removed in vacuo first on a rotovap and then on a high vacuum line (4 Pa, 25° C.) for 2 hr. The product was a viscous light yellow oil (76.0 g, 64% yield).

¹⁹F NMR (DMSO-d₆) δ −124.7. (dt, J_{FH}=6 Hz, J_{FF}=6 Hz, 2F); −138.4 (dt, J_{FH}=53 Hz, 2F). 1H NMR (DMSO-d₆) δ 1.3 (t, J=7.3 Hz, 3H); 3.7 (s, 3H); 4.0 (q, J=7.3 Hz, 2H); 6.1 (tt, J_{FH}=53 Hz, J_{FH}=6 Hz, 1H); 7.2 (s, 1H); 7.3 (s, 1H); 8.5 (s, 1H). % Water by Karl-Fisher titration: 0.18%. Analytical calculation for C₈H₁₂N₂O₃F₄S: C, 32.9: H, 4.1: N, 9.6 Found: C, 33.3: H, 3.7: N, 9.6. Mp 45-46° C. TGA (air): 10% wt. loss @ 379° C., 50% wt. loss @ 420° C. TGA (N₂): 10% wt. loss @ 378° C., 50% wt. loss @ 418° C. The reaction scheme is shown below:

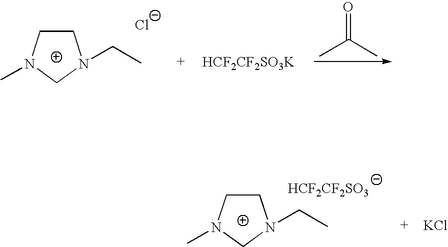

### (J) Synthesis of 1-ethyl-3-methylimidazolium1,1,2,3,3,3-hexafluoropropanesulfonate (Emim-HFPS)

To a 1 liter round bottom flask was added 1-ethyl-3-methylimidazolium chloride (Emim-Cl, 98%, 50.5 g) and reagent grade acetone (400 mL). The mixture was gently warmed (50° C.) until almost all of the Emim-Cl dissolved. To a separate 500 mL flask was added potassium 1,1,2,3,3,3-hexafluoropropanesulfonate (HFPS-K, 92.2 g) along with reagent grade acetone (300 mL). This second mixture was stirred magnetically at room temperature until all of the HFPS-K dissolved.

These solutions were combined and stirred under positive N₂ pressure at 26° C. for 12 hr producing a milky white suspension. The KCI precipitate was allowed to settle overnight leaving a clear yellow solution above it.

The reaction mixture was filtered once through a celite/acetone pad and again through a fritted glass funnel. The acetone was removed in vacuo first on a rotovap and then on a high vacuum line (4 Pa, 25° C.) for 2 hr. The product was a viscous light yellow oil (103.8 g, 89% yield).

¹⁹F NMR (DMSO-d₆) δ −73.8 (s, 3F); −114.5, −121.0 (ABq, J=258 Hz, 2F); −210.6 (m, 1F, J_{HF}=41.5 Hz). ¹H NMR (DMSO-d₆) δ 1.4 (t, J=7.3 Hz, 3H); 3.9 (s, 3H); 4.2 (q, J=7.3 Hz, 2H,); 5.8 (m, J_{HF}=41.5 Hz, 1H,); 7.7 (s, 1H); 7.8 (s, 1H); 9.1 (s, 1H). % Water by Karl-Fisher titration: 0.12%. Analytical calculation for C₉H₁₂N₂O₃F₆S: C, 31.5: H, 3.5: N, 8.2. Experimental Results: C, 30.9: H, 3.3: N, 7.8. TGA (air): 10% wt. loss @ 342° C., 50% wt. loss @ 373° C. TGA (N₂): 10% wt. loss @ 341° C., 50% wt. loss @ 374° C. The reaction scheme is shown below:

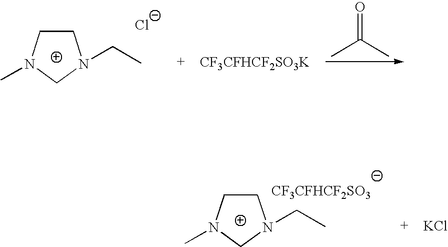

### (K) Synthesis of 1-hexyl-3-methylimidazolium1,1,2,2-tetrafluoroethanesulfonate

1-Hexyl-3-methylimidazolium chloride (10 g, 0.0493 moles) was mixed with reagent-grade acetone (100 mL) in a large round-bottomed flask and stirred vigorously under a nitrogen blanket. Potassium 1,1,2,2-tetrafluoroethane sulfonate (TFES-K, 10 g, 0.0455 moles) was added to reagent grade acetone (100 mL) in a separate round-bottomed flask, and this solution was carefully added to the 1-hexyl-3-methylimidazolium chloride/acetone mixture. The mixture was left to stir overnight. The reaction mixture was then filtered using a large frit glass funnel to remove the white KCl precipitate formed, and the filtrate was placed on a rotary evaporator for 4 hours to remove the acetone.

Appearance: pale yellow, viscous liquid at room temperature. ¹H NMR (DMSO-d₆): δ 0.9 (t, 3H); 1.3 (m, 6H); 1.8 (m, 2H); 3.9 (s, 3H); 4.2 (t, 2H); 6.4 (tt, 1H); 7.7(s, 1H); 7.8 (s, 1H); 9.1 (s, 1H). % Water by Karl-Fischer titration: 0.03% TGA (air): 10% wt. loss @ 365° C., 50% wt. loss @ 410° C. TGA (N₂): 10% wt. loss @ 370° C., 50% wt. loss @ 415° C. The reaction scheme is shown below:

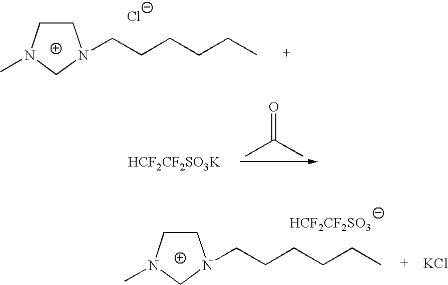

### (L) Synthesis of 1-dodecyl-3-methylimidazolium1,1,2,2-tetrafluoroethanesulfonate

1-Dodecyl-3-methylimidazolium chloride (34.16 g, 0.119 moles) was partially dissolved in reagent-grade acetone (400 mL) in a large round-bottomed flask and stirred vigorously. Potassium 1,1,2,2-tetrafluoroethanesulfonate (TFES-K, 26.24 g, 0.119 moles) was added to reagent grade acetone (400 mL) in a separate round-bottomed flask, and this solution was carefully added to the 1-dodecyl-3-methylimidazolium chloride solution. The reaction mixture was heated at 60° C. under reflux for approximately 16 hours. The reaction mixture was then filtered using a large frit glass funnel to remove the white KCl precipitate formed, and the filtrate was placed on a rotary evaporator for 4 hours to remove the acetone.

¹H NMR (CD₃CN): δ 0.9 (t, 3H); 1.3 (m. 18H); 1.8 (m, 2H); 3.9 (s, 3H); 4.2 (t, 2H); 6.4 (tt, 1H); 7.7(s, 1H); 7.8 (s, 1H); 9.1 (s, 1H). ¹⁹F NMR (CD₃CN): δ −125.3 (m, 2F); −137 (dt, 2F). % Water by Karl-Fischer titration : 0.24% TGA (air): 10% wt. loss @ 370° C., 50% wt. loss @ 410° C. TGA (N₂): 10% wt. loss @ 375° C., 50% wt. loss @ 410° C. The reaction scheme is shown below:

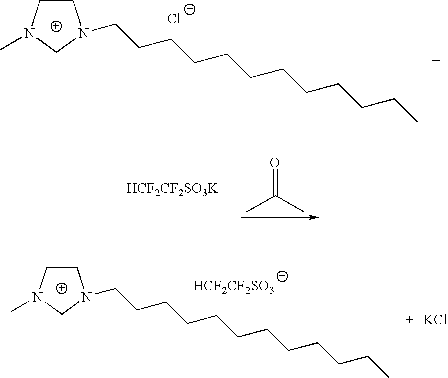

### (M) Synthesis of 1-hexadecyl-3-methylimidazolium1,1,2,2-tetrafluoroethanesulfonate

1-Hexadecyl-3-methylimidazolium chloride (17.0 g, 0.0496 moles) was partially dissolved in reagent-grade acetone (100 mL) in a large round-bottomed flask and stirred vigorously. Potassium 1,1,2,2-tetrafluoroethanesulfonate (TFES-K, 10.9 g, 0.0495 moles) was added to reagent grade acetone (100 mL) in a separate round-bottomed flask, and this solution was carefully added to the 1-hexadecyl-3-methylimidazolium chloride solution. The reaction mixture was heated at 60° C. under reflux for approximately 16 hours. The reaction mixture was then filtered using a large frit glass funnel to remove the white KCl precipitate formed, and the filtrate was placed on a rotary evaporator for 4 hours to remove the acetone.

Appearance: white solid at room temperature. ¹H NMR (CD₃CN): δ 0.9 (t, 3H); 1.3 (m, 26H); 1.9 (m, 2H); 3.9 (s, 3H); 4.2 (t, 2H ); 6.3 (tt, 1H); 7.4 (s, 1H); 7.4 (s, 1H); 8.6 (s, 1H). ¹⁹F NMR (CD₃CN): δ −125.2 (m, 2F); −136.9 (dt, 2F). % Water by Karl-Fischer titration : 200 ppm. TGA (air): 10% wt. loss @ 360° C., 50% wt. loss @ 395° C. TGA (N₂): 10% wt. loss @ 370° C., 50% wt. loss @ 400° C. The reaction scheme is shown below:

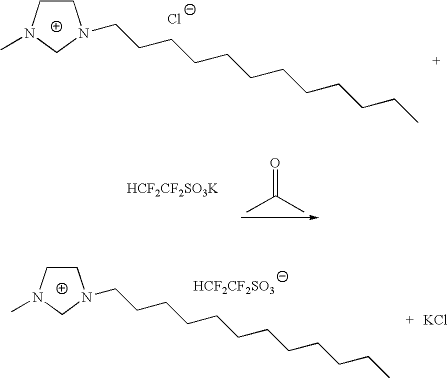

### (N) Synthesis of 1-octadecyl-3-methylimidazolium1,1,2,2-tetrafluoroethanesulfonate

1-Octadecyl-3-methylimidazolium chloride (17.0 g, 0.0458 moles) was partially dissolved in reagent-grade acetone (200 mL) in a large round-bottomed flask and stirred vigorously. Potassium 1,1,2,2-tetrafluoroethanesulfonate (TFES-K, 10.1 g, 0.0459 moles), was added to reagent grade acetone (200 mL) in a separate round-bottomed flask, and this solution was carefully added to the 1-octadecyl-3-methylimidazolium chloride solution. The reaction mixture was heated at 60° C. under reflux for approximately 16 hours. The reaction mixture was then filtered using a large frit glass funnel to remove the white KCl precipitate formed, and the filtrate was placed on a rotary evaporator for 4 hours to remove the acetone.

¹H NMR (CD₃CN): δ 0.9 (t, 3H); 1.3 (m, 30H); 1.9 (m, 2H); 3.9 (s, 3H); 4.1 (t, 2H); 6.3 (tt, 1H); 7.4(s, 1H); 7.4 (s, 1H); 8.5 (s, 1H). ¹⁹F NMR (CD₃CN): δ −125.3 (m, 2F); −136.9 (dt, 2F). % Water by Karl-Fischer titration: 0.03%. TGA (air): 10% wt. loss @ 360° C., 50% wt. loss @ 400° C. TGA (N₂): 10% wt. loss @ 365° C., 50% wt. loss @ 405° C. The reaction scheme is shown below:

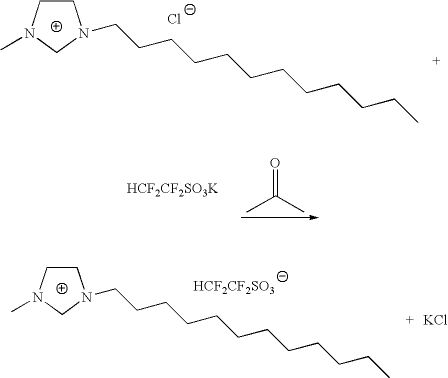

### (O) Synthesis of N-(1,1,2,2-tetrafluoroethyl)propylimidazole1,1,2,2-tetrafluoroethanesulfonate

Imidazole (19.2 g) was added to of tetrahydrofuran (80 mLs). A glass shaker tube reaction vessel was filled with the THF-containing imidazole solution. The vessel was cooled to 18 degrees C., evacuated to 0.08 MPa, and purged with nitrogen. The evacuate/purge cycle was repeated two more times. Tetrafluoroethylene (TFE, 5 g) was then added to the vessel, and it was heated to 100° C., at which time the inside pressure was about 0.72 MPa. As the TFE pressure decreased due to the reaction, more TFE was added in small aliquots (5 g each) to maintain operating pressure roughly between 0.34 MPa and 0.86 MPa. Once 40 g of TFE had been fed, the vessel was vented and cooled to 25° C. The THF was then removed under vacuum and the product was vacuum distilled at 40° C. to yield pure product as shown by ¹H and ¹⁹F NMR (yield 44 g). Iodopropane (16.99 g) was mixed with 1-(1,1,2,2-tetrafluoroethyl)imidazole (16.8 g) in dry acetonitrile (100 mL), and the mixture was refluxed for 3 days. The solvent was removed in vacuo, yielding a yellow waxy solid (yield 29 g). The product, 1 -propyl-3-(1,1,2,2-tetrafluoroethyl)imidazolium iodide was confirmed by ¹H NMR (in d acetonitrile) [ 0.96 (t, 3H); 1.99 (m, 2H); 4.27 (t, 2H); 6.75 (t, 1 H); 7.72 (d, 2H); 9.95 (s, 1H)].

Iodide (24 g) was then added to 60 mL of dry acetone, followed by 15.4 g of potassium 1,1,2,2-tetrafluoroethanesulfonate in 75 mL of dry acetone. The mixture was heated at 60° C. overnight and a dense white precipitate was formed (potassium iodide). The mixture was cooled, filtered, and the solvent from the filtrate was removed using a rotary evaporator. Some further potassium iodide was removed under filtration. The product was further purified by adding 50 g of acetone, 1 g of charcoal, 1 g of celite and 1 g of silica gel. The mixture was stirred for 2 hours, filtered and the solvent removed. This yielded 15 g of a liquid, shown by NMR to be the desired product.

### (P) Synthesis of 1-butyl-3-methylimidazolium1,1,2,3,3,3-hexafluoropropanesulfonate (Bmim-HFPS)

1-Butyl-3-methylimidazolium chloride (Bmim-Cl, 50.0 g) and high purity dry acetone (>99.5%, 500 mL) were combined in a 1 liter flask and warmed to reflux with magnetic stirring until the solid all dissolved. At room temperature in a separate 1 liter flask, potassium-1,1,2,3,3,3-hexafluoropropanesulfonte (HFPS-K) was dissolved in high purity dry acetone (550 mL). These two solutions were combined at room temperature and allowed to stir magnetically for 12 hr under positive nitrogen pressure. The stirring was stopped, and the KCl precipitate was allowed to settle. This solid was removed by suction filtration through a fritted glass funnel with a celite pad. The acetone was removed in vacuo to give a yellow oil. The oil was further purified by diluting with high purity acetone (100 mL) and stirring with decolorizing carbon (5 g). The mixture was suction filtered and the acetone removed in vacuo to give a colorless oil. This was further dried at 4 Pa and 25° C. for 2 hr to provide 68.6 g of product.

¹⁹F NMR (DMSO-d₆) δ −73.8.(s, 3F); −114.5, −121.0 (ABq, J=258 Hz, 2F); −210.6 (m, J=42 Hz, 1F). ¹H NMR (DMSO-d₆) δ 0.9 (t, J=7.4 Hz, 3H); 1.3 (m, 2H); 1.8 (m, 2H); 3.9 (s, 3H); 4.2 (t, J=7 Hz, 2H); 5.8 (dm, J=42 Hz, 1H); 7.7 (s, 1H); 7.8 (s, 1H); 9.1 (s, 1H). % Water by Karl-Fisher titration: 0.12%. Analytical calculation for C₉H₁₂F₆N₂O₃S: C, 35.7: H, 4.4: N, 7.6. Experimental Results: C, 34.7: H, 3.8: N, 7.2. TGA (air): 10% wt. loss @ 340° C., 50% wt. loss @ 367° C. TGA (N₂): 10% wt. loss @ 335° C., 50% wt. loss @ 361° C. Extractable chloride by ion chromatography: 27 ppm.

### (Q) Synthesis of 1-butyl-3-methylimidazolium1,1,2-trifluoro-2-(trifluoromethoxy)ethanesulfonate (Bmim-TTES)

1-Butyl-3-methylimidazolium chloride (Bmim-Cl, 10.0 g) and deionized water (15 mL) were combined at room temperature in a 200 mL flask. At room temperature in a separate 200 mL flask, potassium 1,1,2-trifluoro-2-(trifluoromethoxy)ethanesulfonate (TTES-K, 16.4 g) was dissolved in deionized water (90 mL). These two solutions were combined at room temperature and allowed to stir magnetically for 30 min. under positive nitrogen pressure to give a biphasic mixture with the desired ionic liquid as the bottom phase. The layers were separated, and the aqueous phase was extracted with 2×50 mL portions of methylene chloride. The combined organic layers were dried over magnesium sulfate and concentrated in vacuo. The colorless oil product was dried at for 4 hr at 5 Pa and 25° C. to afford 15.0 g of product.

¹⁹F NMR (DMSO-d₆) δ −56.8 (d, J_{FH}=4 Hz, 3F); −119.5, −119.9 (subsplit ABq, J=260 Hz, 2F); −142.2 (dm, J_{FH}=53 Hz, 1F). ¹H NMR (DMSO-d₆) δ 0.9 (t, J=7.4 Hz, 3H); 1.3 (m, 2H); 1.8 (m, 2H); 3.9 (s, 3H); 4.2 (t, J=7.0 Hz, 2H); 6.5 (dt, J=53 Hz, J=7 Hz, 1H); 7.7 (s, 1H); 7.8 (s, 1H); 9.1 (s, 1H). % Water by Karl-Fisher titration: 613 ppm. Analytical calculation for C11H16F6N2O4S: C, 34.2: H, 4.2: N, 7.3. Experimental Results: C, 34.0: H, 4.0: N, 7.1. TGA (air): 10% wt. loss @ 328° C., 50% wt. loss @ 354° C. TGA (N₂): 10% wt. loss @ 324° C., 50% wt. loss @ 351° C. Extractable chloride by ion chromatography: <2 ppm.

### (R) Synthesis of 1-butyl-3-methylimidazolium1,1,2-trifluoro-2-(perfluoroethoxy)ethanesulfonate (Bmim-TPES)

1-Butyl-3-methylimidazolium chloride (Bmim-Cl, 7.8 g) and dry acetone (150 mL) were combined at room temperature in a 500 mL flask. At room temperature in a separate 200 mL flask, potassium 1,1,2-trifluoro-2-(perfluoroethoxy)ethanesulfonate (TPES-K, 15.0 g) was dissolved in dry acetone (300 mL). These two solutions were combined and allowed to stir magnetically for 12 hr under positive nitrogen pressure. The KCl precipitate was then allowed to settle leaving a colorless solution above it. The reaction mixture was filtered once through a celite/acetone pad and again through a fritted glass funnel to remove the KCl. The acetone was removed in vacuo first on a rotovap and then on a high vacuum line (4 Pa, 25° C.) for 2 hr. Residual KCCI was still precipitating out of the solution, so methylene chloride (50 mL) was added to the crude product which was then washed with deionized water (2×50 mL). The solution was dried over magnesium sulfate, and the solvent was removed in vacuo to give the product as a viscous light yellow oil (12.0 g, 62% yield).

¹⁹F NMR (CD₃CN) δ −85.8 (s, 3F); −87.9, −90.1 (subsplit ABq, J_{FF}=147 Hz, 2F); −120.6, −122.4 (subsplit ABq, J_{FF}=258 Hz, 2F); −142.2 (dm, J_{FH}=53 Hz, 1F). ¹H NMR (CD₃CN) δ 1.0 (t, J=7.4 Hz, 3H); 1.4 (m, 2H); 1.8 (m, 2H); 3.9 (s, 3H); 4.2 (t, J=7.0 Hz, 2H); 6.5 (dm, J=53 Hz, 1H); 7.4 (s, 1H); 7.5 (s, 1H); 8.6 (s, 1H). % Water by Karl-Fisher titration: 0.461. Analytical calculation for C12H16F8N2O4S: C, 33.0: H, 3.7. Experimental Results: C, 32.0: H, 3.6. TGA (air): 10% wt. loss @ 334° C., 50% wt. loss @ 353° C. TGA (N₂): 10% wt. loss @ 330° C., 50% wt. loss @ 365° C.

### (S) Synthesis of tetradecyl(tri-n-butyl)phosphonium1,1,2,3,3,3-hexafluoropropanesulfonate ([4.4.4.14]P-HFPS)

To a 4 l round bottomed flask was added the ionic liquid tetradecyl(tri-n-butyl)phosphonium chloride (Cyphos® IL 167, 345 g) and deionized water (1000 mL). The mixture was magnetically stirred until it was one phase. In a separate 2 liter flask, potassium 1,1,2,3,3,3-hexafluoropropanesulfonate (HFPS-K, 214.2 g) was dissolved in deionized water (1100 mL). These solutions were combined and stirred under positive N₂ pressure at 26° C. for 1 hr producing a milky white oil. The oil slowly solidified (439 g) and was removed by suction filtration and then dissolved in chloroform (300 mL). The remaining aqueous layer (pH=2) was extracted once with chloroform (100 mL). The chloroform layers were combined and washed with an aqueous sodium carbonate solution (50 mL) to remove any acidic impurity. They were then dried over magnesium sulfate, suction filtered, and reduced in vacuo first on a rotovap and then on a high vacuum line (4 Pa, 100° C.) for 16 hr to yield the final product as a white solid (380 g, 76% yield).

¹⁹F NMR (DMSO-d6) δ −73.7 (s, 3F); −114.6, −120.9 (ABq, J=258 Hz, 2F); −210.5 (m, J_{HF}=41.5 Hz, 1F). ¹H NMR (DMSO-d₆) δ 0.8 (t, J=7.0 Hz, 3H); 0.9 (t, J=7.0 Hz, 9H); 1.3 (br s, 20H); 1.4 (m, 16H); 2.2 (m, 8H); 5.9 (m, J_{HF}=42 Hz, 1H). % Water by Karl-Fisher titration: 895 ppm. Analytical calculation for C29H57F6O3PS: C, 55.2: H, 9.1: N, 0.0. Experimental Results: C, 55.1: H, 8.8: N, 0.0. TGA (air): 10% wt. loss @ 373° C., 50% wt. loss @ 421° C. TGA (N₂): 10% wt. loss @ 3830° C., 50% wt. loss @ 436° C.

### (T) Synthesis of Tetradecyl(tri-n-hexyl)phosphonium1,1,2-trifluoro-2-(perfluoroethoxy)ethanesulfonate ([6.6.6.14]P-TPES)

To a 500 mL round bottomed flask was added acetone (Spectroscopic grade, 50 mL) and ionic liquid tetradecyl(tri-n-hexyl)phosphonium chloride (Cyphos® IL 101, 33.7 g). The mixture was magnetically stirred until it was one phase. In a separate 1 liter flask, potassium 1,1,2-trifluoro-2-(perfluoroethoxy)ethanesulfonate (TPES-K, 21.6 g) was dissolved in acetone (400 mL). These solutions were combined and stirred under positive N₂ pressure at 26° C. for 12 hr producing a white precipitate of KCl. The precipitate was removed by suction filtration, and the acetone was removed in vacuo on a rotovap to produce the crude product as a cloudy oil (48 g). Chloroform (100 mL) was added, and the solution was washed once with deionized water (50 mL). It was then dried over magnesium sulfate and reduced in vacuo first on a rotovap and then on a high vacuum line (8 Pa, 24° C.) for 8 hr to yield the final product as a slightly yellow oil (28 g, 56% yield).

¹⁹F NMR (DMSO-d₆) δ −86.1 (s, 3F); −88.4, −90.3 (subsplit ABq, J_{FF}=147 Hz, 2F); −121.4, −122.4 (subsplit ABq, J_{FF}=258 Hz, 2F); −143.0 (dm, J_{FH}=53 Hz, 1F). ¹H NMR (DMSO-d₆) δ 0.9 (m, 12H); 1.2 (m, 16H); 1.3 (m, 16H); 1.4 (m, 8H); 1.5 (m, 8H); 2.2 (m, 8H); 6.3 (dm, J_{FH}=54 Hz, 1H). % Water by Karl-Fisher titration: 0.11. Analytical calculation for C36H69F8O4PS: C, 55.4: H, 8.9: N, 0.0. Experimental Results: C, 55.2: H, 8.2: N, 0.1. TGA (air): 10% wt. loss @ 31 1 ° C., 50% wt. loss @ 339° C. TGA (N₂): 10% wt. loss @ 315° C., 50% wt. loss @ 343° C.

### (U) Synthesis of tetradecyl(tri-n-hexyl)phosphonium1,1,2-trifluoro-2-(trifluoromethoxy)ethanesulfonate ([6.6.6.14]P-TTES)

To a 100 mL round bottomed flask was added acetone (Spectroscopic grade, 50 mL) and ionic liquid tetradecyl(tri-n-hexyl)phosphonium chloride (Cyphos® IL 101, 20.2 g). The mixture was magnetically stirred until it was one phase. In a separate 100 mL flask, potassium 1,1,2-trifluoro-2-(trifluoromethoxy)ethanesulfonate (TTES-K, 11.2 g) was dissolved in acetone (100 mL). These solutions were combined and stirred under positive N₂ pressure at 26° C. for 12 hr producing a white precipitate of KCl.

The precipitate was removed by suction filtration, and the acetone was removed in vacuo on a rotovap to produce the crude product as a cloudy oil. The product was diluted with ethyl ether (100 mL) and then washed once with deionized water (50 mL), twice with an aqueous sodium carbonate solution (50 mL) to remove any acidic impurity, and twice more with deionized water (50 mL). The ether solution was then dried over magnesium sulfate and reduced in vacuo first on a rotovap and then on a high vacuum line (4 Pa, 24° C.) for 8 hr to yield the final product as an oil (19.0 g, 69% yield).

¹⁹F NMR (CD₂Cl₂) δ −60.2.(d, J_{FH}=4 Hz, 3F); −120.8, −125.1 (subsplit ABq, J=260 Hz, 2F); −143.7 (dm, J_{FH}=53 Hz, 1F). ¹H NMR (CD₂Cl₂) δ 0.9 (m, 12H); 1.2 (m, 16H); 1.3 (m, 16H); 1.4 (m, 8H); 1.5 (m, 8H); 2.2 (m, 8H); 6.3 (dm, J_{FH}=54 Hz, 1H). % Water by Karl-Fisher titration: 412 ppm. Analytical calculation for C35H69F6O4PS: C, 57.5: H, 9.5: N, 0.0. Experimental results: C, 57.8: H, 9.3: N, 0.0. TGA (air): 10% wt. loss @ 331° C., 50% wt. loss @ 359° C. TGA (N₂): 10% wt. loss @ 328° C., 50% wt. loss @ 360° C.

### (V) Synthesis of 1-ethyl-3-methylimidazolium1,1,2,2-tetrafluoro-2-(pentafluoroethoxy)sulfonate (Emim-TPENTAS)

To a 500 mL round bottomed flask was added 1-ethyl-3-methylimidazolium chloride (Emim-Cl, 98%, 18.0 g) and reagent grade acetone (150 mL). The mixture was gently warmed (50° C.) until all of the Emim-Cl dissolved. In a separate 500 mL flask, potassium 1,1,2,2-tetrafluoro-2-(pentafluoroethoxy)sulfonate (TPENTAS-K, 43.7 g) was dissolved in reagent grade acetone (450 mL).

These solutions were combined in a 1 liter flask producing a white precipitate (KCl). The mixture was stirred at 24° C. for 8 hr. The KCl precipitate was then allowed to settle leaving a clear yellow solution above it. The KCl was removed by filtration through a celite/acetone pad. The acetone was removed in vacuo to give a yellow oil which was then diluted with chloroform (100 mL). The chloroform was washed three times with deionized water (50 mL), dried over magnesium sulfate, filtered, and reduced in vacuo first on a rotovap and then on a high vacuum line (4 Pa, 25° C.) for 8 hr. The product was a light yellow oil (22.5 g). ¹⁹F NMR (DMSO-d₆) δ −82.9.(m, 2F); −87.3 (s, 3F); −89.0 (m, 2F); −118.9 (s, 2F). ¹H NMR (DMSO-d₆) δ 1.5 (t, J=7.3 Hz, 3H); 3.9 (s, 3H); 4.2 (q, J=7.3 Hz, 2H); 7.7 (s, 1H); 7.8 (s, 1H); 9.1 (s, 1H). % Water by Karl-Fisher titration: 0.17%. Analytical calculation for C10H11N2O4F9S: C, 28.2: H, 2.6: N, 6.6 Experimental results: C, 28.1: H, 2.9: N, 6.6. TGA (air): 10% wt. loss @ 351° C., 50% wt. loss @ 401° C. TGA (N₂): 10% wt. loss @ 349° C., 50% wt. loss @ 406° C.

### (W) Synthesis of tetrabutylphosphonium1,1,2-trifluoro-2-(perfluoroethoxy)ethanesulfonate (TBP-TPES)

To a 200 mL round bottomed flask was added deionized water (100 mL) and tetra-n-butylphosphonium bromide (Cytec Canada Inc., 20.2 g). The mixture was magnetically stirred until the solid all dissolved. In a separate 300 mL flask, potassium 1,1,2-trifluoro-2-(perfluoroethoxy)ethanesulfonate (TPES-K, 20.0 g) was dissolved in deionized water (400 mL) heated to 70° C. These solutions were combined and stirred under positive N2 pressure at 26° C. for 2 hr producing a lower oily layer. The product oil layer was separated and diluted with chloroform (30 mL), then washed once with an aqueous sodium carbonate solution (4 mL) to remove any acidic impurity, and three times with deionized water (20 mL). It was then dried over magnesium sulfate and reduced in vacuo first on a rotovap and then on a high vacuum line (8 Pa, 24° C.) for 2 hr to yield the final product as a colorless oil (28.1 g, 85% yield).

¹⁹F NMR (CD₂Cl₂) δ −86.4 (s, 3F); −89.0, −90.8 (subsplit ABq, J_{FF}=147 Hz, 2F); −119.2, −125.8 (subsplit ABq, J_{FF}=254Hz, 2F); −141.7 (dm, J_{FH}=53Hz, 1F). ¹H NMR (CD₂Cl₂) δ 1.0 (t, J=7.3 Hz, 12H);1.5 (m, 16H); 2.2 (m, 8H); 6.3 (dm, J_{FH}=54 Hz, 1H). % Water by Karl-Fisher titration: 0.29. Analytical calculation for C20H37F8O4PS: C, 43.2: H, 6.7: N, 0.0. Experimental results: C, 42.0: H, 6.9: N, 0.1. Extractable bromide by ion chromatography: 21 ppm.

### (X) Synthesis of (3,3,4,4,5,5,6,6,7,7,8,8,8-tridecafluorooctyl)-trioctylphosphonium1,1,2,2-tetrafluoroethanesulfonate

Trioctyl phosphine (31 g) was partially dissolved in reagent-grade acetonitrile (250 mL) in a large round-bottomed flask and stirred vigorously. 1,1,1,2,2,3,3,4,4,5,5,6,6-Tridecafluoro-8-iodooctane (44.2 g) was added, and the mixture was heated under reflux at 110° C. for 24 hours. The solvent was removed under vacuum giving (3,3,4,4,5,5,6,6,7,7,8,8,8-tridecafluorooctyl)-trioctylphosphonium iodide as a waxy solid (30.5 g). Potassium 1,1,2,2-tetrafluoroethanesulfonate (TFES-K, 13.9 g) was dissolved in reagent grade acetone (100 mL) in a separate round-bottomed flask, and to this was added (3,3,4,4,5,5,6,6,7,7,8,8,8-tridecafluorooctyl)-trioctylphosphonium iodide (60 g). The reaction mixture was heated at 60° C. under reflux for approximately 16 hours. The reaction mixture was then filtered using a large frit glass funnel to remove the white KI precipitate formed, and the filtrate was placed on a rotary evaporator for 4 hours to remove the acetone. The liquid was left for 24 hours at room temperature and then filtered a second time (to remove KI) to yield the product (62 g) as shown by proton NMR.

### (Y) Synthesis of 1-methyl-3-(3,3,4,4,5,5,6,6,7,7,8,8,8-tridecafluorooctyl)imidazolium1,1,2,2-tetrafluoroethanesulfonate

1-Methylimidazole (4.32 g, 0.52 mol) was partially dissolved in reagent-grade toluene (50 mL) in a large round-bottomed flask and stirred vigorously. 1,1,1,2,2,3,3,4,4,5,5,6,6-Tridecafluoro-8-iodooctane (26 g, 0.053 mol) was added, and the mixture was heated under reflux at 110° C. for 24 hours. The solvent was removed under vacuum giving 1-methyl-3-(3,3,4,4,5,5,6,6,7,7,8,8,8-tridecafluorooctyl)imidazolium iodide (30.5 g) as a waxy solid. Potassium 1,1,2,2-tetrafluoroethanesulfonate (TFES-K, 12 g) was added to reagent grade acetone (100 mL) in a separate round-bottomed flask, and this solution was carefully added to the 1-methyl-3-(3,3,4,4,5,5,6,6,7,7,8,8,8-tridecafluorooctyl)imidazolium iodide which had been dissolved in acetone (50 mL). The reaction mixture was heated under reflux for approximately 16 hours. The reaction mixture was then filtered using a large frit glass funnel to remove the white KI precipitate formed, and the filtrate was placed on a rotary evaporator for 4 hours to remove the acetone. The oily liquid was then filtered a second time to yield the product, as shown by proton NMR.

### Example 1

### Isomerization of 1-dodecene in the Presence of the Ionic Liquid 1-dodecyl-3-methylimidazolium 1,1,2,2-tetrafluoroethanesulfonate

The ionic liquid 1-dodecyl-3-methylimidazolium 1,1,2,2-tetrafluoroethanesulfonate (Ddmim-TFES; 2.0 g) was weighed into a small round-bottomed flask, and the flask was dried overnight at 150° C. under vacuum. The flask was removed from the oven, quickly stoppered, and allowed to cool in the antechamber of a dry box under vacuum before being transported into the dry box. HCF₂CF₂SO₃H (0.5 g) and 1-dodecene (30 mL) were added to the round bottomed flask in the dry box. The flask was then lowered into an oil bath and heated for 2 hours at 100 ° C. with stirring.

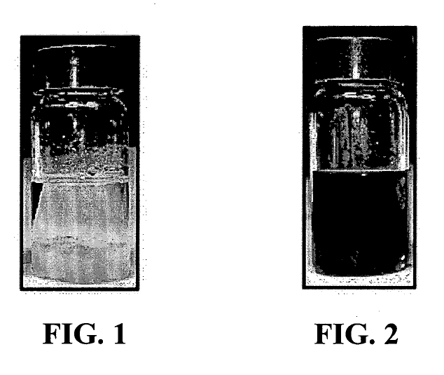

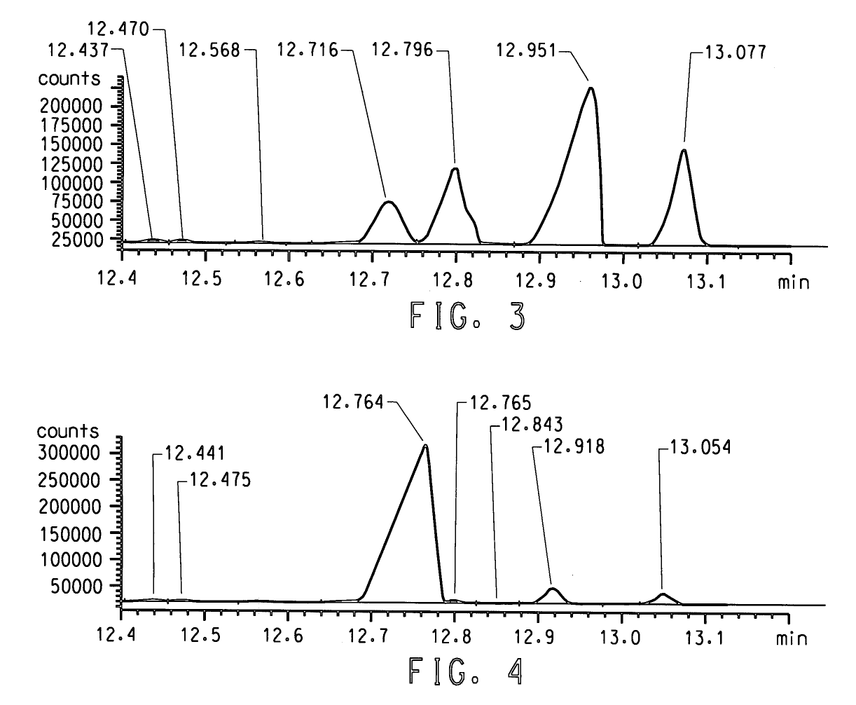

Upon completion of the reaction, the ionic liquid and acid formed a separate phase at the bottom, with the product in the top phase, as shown in FIG. 1 (after the material was decanted to a vial). The product is colorless, i.e. water-white. The GC trace of the product phase after 2 hours is shown in FIG. 3; GC analysis confirmed the conversion of the 1-dodecene to the equilibrium isomers, with less than 20% of the 1-dodecene remaining.

### Example 2

### (Comparative Example): Isomerization of 1-dodecene in the Absence of an Ionic Liquid

A small round-bottomed flask was dried overnight at 150° C. under vacuum, removed from the oven, quickly stoppered, and allowed to cool in the antechamber of a dry box under vacuum before being transported into the dry box. HCF₂CF₂SO₃H (0.5 g) and 1-dodecene (30 mL) were added to the round bottomed flask in the dry box. The flask was then lowered into an oil bath and heated for 2 hours at 100 ° C. with stirring. The GC trace obtained after 2 hours is shown in FIG. 4; GC analysis showed that less than 5% of the 1-dodecene had reacted. Only one phase was observed after the reaction (see FIG. 2). The color of the solution after the reaction was deep red; depending on the intended use of the product, color formation is often not desirable.

### Example 3

### Isomerization of 1-dodecene in the Presence of the Ionic Liquid 1-octadecyl-3-methylimidazolium1,1,2,2-tetrafluoroethanesulfonate

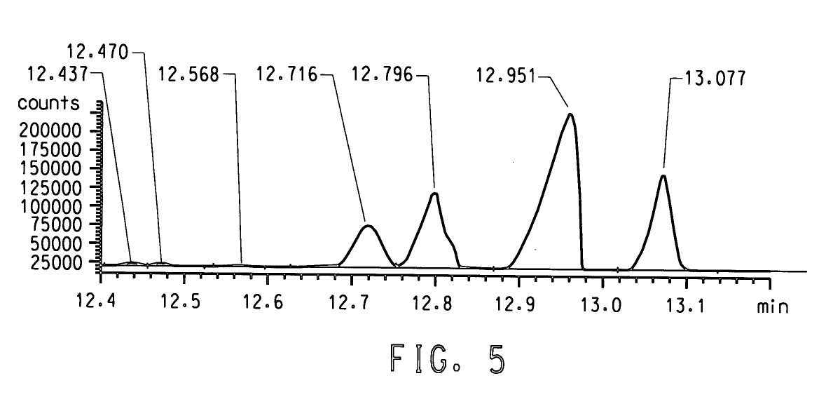

The ionic liquid 1-octadecyl-3-methylimidazolium1,1,2,2-tetrafluoroethanesulfonate (Odmim-TFES; 2.0 g) was weighed into a small round-bottomed flask, and the flask was dried overnight at 150° C. under vacuum. The flask was removed from the oven, quickly stoppered, and allowed to cool in the antechamber of a dry box under vacuum before being transported into the dry box. HCF₂CF₂SO₃H (0.5 g) and 1-dodecene (30 mL) were added to the round bottomed flask in the dry box. The flask was then lowered into an oil bath and heated for two hours at 100° C. with stirring. The GC trace of the colorless product phase after 2 hours is shown in FIG. 5; GC analysis confirmed the conversion of the 1-dodecene to the equilibrium isomers, with less than 20% of the 1-dodecene remaining. Upon completion of the reaction, the ionic liquid and acid formed a separate phase at the bottom, with the product in the top phase.

1. A process for making internal olefins comprising:
(A) forming a reaction mixture comprising (1) at least one α-olefin having from 4 to 25 carbons, (2) at least one acid catalyst selected from the group consisting of rare earth fluorinated alkyl sulfonates, organic sulfonic acids, fluoroalkyl sulfonic acids, metal sulfonates, metal trifluoroacetates, and combinations thereof, and (3) at least one ionic liquid having the Formula Z⁺A⁻, wherein Z⁺ is a cation selected from the group consisting of: 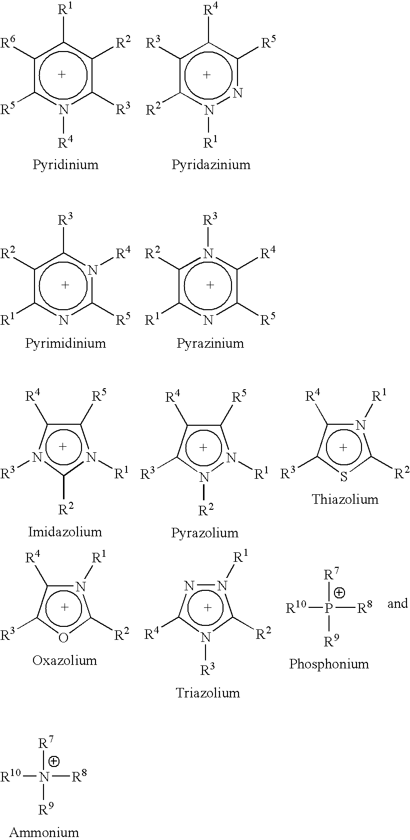
wherein R¹, R², R³, R⁴, R⁵and R⁶ are independently selected from the group consisting of:
(i) H
(ii) halogen
(iii) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
(iv) —CH₃, —C₂H₅, or C₃ to C₂₅ straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
(v) C₆ to C₂₅ unsubstituted aryl or unsubstituted heteroaryl having one to three heteroatoms independently selected from the group consisting of O, N and S; and
(vi) C₆ to C₂₅ substituted aryl or substituted heteroaryl having one to three heteroatoms independently selected from the group consisting of O, N and S; and wherein said substituted aryl or substituted heteroaryl has one to three substituents independently selected from the group consisting of
(1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
(2) OH,
(3) NH₂, and
(4) SH;
R⁷, R⁸, R⁹, and R¹⁰ are independently selected from the group consisting of:
(vii) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
(viii) —CH₃, —C₂H₅, or C₃ to C₂₅ straight-chain, branched or cyclic alkane or alkene comprising one to three heteroatoms selected from the group consisting of O, N and S, and optionally substituted with at least one member selected from the group consisting of Cl, Br, F, I, OH, NH₂ and SH;
(ix) C₆ to C₂₅ unsubstituted aryl, or C₃ to C₂₅ unsubstituted heteroaryl having one to three heteroatoms independently selected from the group consisting of O, N and S; and
(x) C₆ to C₂₅ substituted aryl, or C₃ to C₂₅ substituted heteroaryl having one to three heteroatoms independently selected from the group consisting of O, N and S; and wherein said substituted aryl or substituted heteroaryl has one to three substituents independently selected from the group consisting of
(1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
(2) OH,
(3) NH₂, and
(4) SH;
wherein optionally at least two of R¹, R², R³, R⁴, R⁵, R⁶, R⁷, R⁸, R⁹, and R¹⁰ can together form a cyclic or bicyclic alkanyl or alkenyl group; and
A⁻ is R¹¹—SO₃— or (R¹²—SO₂)₂N⁻; wherein R¹¹ and R¹² are independently selected from the group consisting of:
(a) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH;
(b) —CH₃, —C₂H₅, or C₃ to C₂₅ straight—chain, branched or cyclic alkane or alkene comprising one to three heteroatoms selected from the group consisting of O, N and S, and optionally substituted with at least one member selected from the group consisting of Cl, Br, F, I, OH, NH₂ and SH;
(c) C₆ to C₂₅ unsubstituted aryl or unsubstituted heteroaryl having one to three heteroatoms independently selected from the group consisting of O, N and S; and
(d) C₆ to C₂₅ substituted aryl or substituted heteroaryl having one to three heteroatoms independently selected from the group consisting of O, N and S; and wherein said substituted aryl or substituted heteroaryl has one to three substituents independently selected from the group consisting of:
(1) —CH₃, —C₂H₅, or C₃ to C₂₅ straight-chain, branched or cyclic alkane or alkene, optionally substituted with at least one member selected from the group consisting of Cl, Br, F, I, OH, NH₂ and SH,
(2) OH,
(3) NH₂, and
(4) SH;
thereby forming an isomer phase comprising at least one internal olefin and an ionic liquid phase that comprises the at least one acid catalyst; and
B) separating the isomer phase from the ionic liquid phase, thereby forming a separated ionic liquid phase.

2. The process of claim 1 wherein Z⁺ is imidazolium or phosphonium.

3. The process of claim 1 wherein A⁻ is selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CF₂CF₂OCFHCF₂SO₃]⁻, [CF₃FHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, [(CF₃CFHCF₂SO₂)₂N]⁻.

4. The process of claim 2 wherein A⁻ is selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CF₂CF₂OCFH₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, [(CF₃CFHCF₂SO₂)₂N]⁻.

5. The process of claim 4 wherein said at least one ionic liquid is selected from the group consisting of 1-butyl-2,3-dimethylimidazolium1,1,2,2-tetrafluoroethanesulfonate, 1-butyl-methylimidazolium1,1,2,2-tetrafluoroethanesulfonate, 1-ethyl-3-methylimidazolium1,1,2,2-tetrafluoroethanesulfonate, 1-ethyl-3-methylimidazolium1,1,2,3,3,3-hexafluoropropanesulfonate, 1-hexyl-3-methylimidazolium1,1,2,2-tetrafluoroethanesulfonate, 1-dodecyl-3-methylimidazolium1,1,2,2-tetrafluoroethanesulfonate, 1-hexadecyl-3-methylimidazolium1,1,2,2-tetrafluoroethanesulfonate, 1-octadecyl-3-methylimidazolium1,1,2,2-tetrafluoroethanesulfonate, 1-propyl-3-(1,1,2,2-tetrafluoroethyl)imidazolium1,1,2,2-tetrafluoroethanesulfonate, 1-(1,1,2,2-tetrafluoroethyl)-3-(3,3,4,4,5,5,6,6,7,7,8,8,8-tridecafluorooctyl)imidazolium1,1,2,2-tetrafluoroethanesulfonate, 1-butyl-3-methylimidazolium1,1,2,3,3,3-hexafluoropropanesulfonate, 1-butyl-3-methylimidazolium1,1,2-trifluoro-2-(trifluoromethoxy)ethanesulfonate, 1-butyl-3-methylimidazolium1,1,2-trifluoro-2-(perfluoroethoxy)ethanesulfonate, 1-butyl-3-methylimidazolium1,1,2-trifluoro-2-(trifluoromethoxy)ethanesulfonate, tetradecyl(tri-n-hexyl)phosphonium1,1,2-trifluoro-2-(perfluoroethoxy)ethanesulfonate, tetradecyl(tri-n-butyl)phosphonium1,1,2,3,3,3-hexafluoropropanesulfonate, tetradecyl(tri-n-hexyl)phosphonium1,1,2-trifluoro-2-(trifluoromethoxy)ethanesulfonate, tetradecyl(tri-n-hexyl)phosphonium1,1,2-trifluoro-2-(perfluoropropoxy)ethanesulfonate, 1-ethyl-3-methylimidazolium1,1,2,2-tetrafluoro-2-(pentafluoroethoxy)sulfonate, (3,3,4,4,5,5,6,6,7,7,8,8,8-tridecafluorooctyl)-trioctylphosphonium1,1,2,2-tetrafluoroethanesulfonate, 1-methyl-3-(3,3,4,4,5,5,6,6,7,7,8,8,8-tridecafluorooctyl)imidazolium1,1,2,2-tetrafluoroethanesulfonate, tetra-n-butylphosphonium1,1,2-trifluoro-2-(trifluoromethoxy)ethanesulfonate, tetra-n-butylphosphonium1,1,2-trifluoro-2-(perfluoroethoxy)ethanesulfonate, and tetra-n-butylphosphonium1,1,2-trifluoro-2-(perfluoropropoxy)ethanesulfonate.

6. The process of claim 1 wherein said at least one acid catalyst is selected from the group consisting of:
(i) bismuth triflate;
(ii) yttrium triflate;
(iii) ytterbium triflate;
(iv) neodymium triflate;
(v) lanthanum triflate;
(vi) scandium triflate;
(vii) zirconium triflate;
(viii) Formula (I); 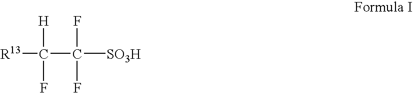
wherein:
R¹³ is selected from the group consisting of:
(1) halogen;
(2) —CH₃, —C₂H₅ or C₃ to C₁₅ straight-chain or branched alkane or alkene, optionally substituted with at least one member selected from the group consisting of Cl, Br, I, OH, NH₂ and SH;
(3) —OCH₃, —OC₂H₅ or C₃ to C₁₅ straight-chain or branched alkoxy, optionally substituted with at least one member selected from the group consisting of Cl, Br, I, OH, NH₂ and SH;
(4) C₁ to C₁₅ straight-chain or branched fluoroalkyl, optionally substituted with at least one member selected from the group consisting of Cl, Br, I, OH, NH₂ and SH;
(5) C₁ to C₁₅ straight-chain or branched fluoroalkoxy, optionally substituted with at least one member selected from the group consisting of Cl, Br, I, OH, NH₂ and SH;
(6) C₁ to C₁₅ straight-chain or branched perfluoroalkyl; and
(7) C₁ to C₁₅ straight-chain or branched perfluoroalkoxy;
(ix) Formula (II) 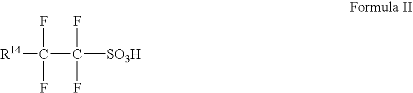
wherein:
R¹⁴ is selected from the group consisting of:
(1) —CH₃, —C₂H₅ or C₃ to C₁₅ straight-chain or branched alkoxy, optionally substituted with at least one member selected from the group consisting of Cl, Br, I, OH, NH₂ and SH;
(2) C₁ to C₁₅ straight-chain or branched fluoroalkoxy, optionally substituted with at least one member selected from the group consisting of Cl, Br, I, OH, NH₂ and SH; and
(3) C₁ to C₁₅ straight-chain or branched perfluoroalkoxy; and
(x) Formula (Ill); 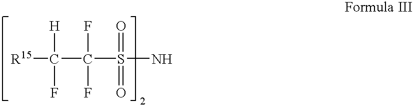
wherein:
R¹⁵ is selected from the group consisting of:
(1) halogen;
(2) —CH₃, —C₂H₅ or C₃ to C₁₅ straight-chain or branched alkane or alkene, optionally substituted with at least one member selected from the group consisting of Cl, Br, I, OH, NH₂ and SH;
(3) —OCH₃, —OC₂H₅ or C₃ to C₁₅ straight-chain or branched alkoxy, optionally substituted with at least one member selected from the group consisting of Cl, Br, I, OH, NH₂ and SH;
(4) C₁ to C₁₅ straight-chain or branched fluoroalkyl, optionally substituted with at least one member selected from the group consisting of Cl, Br, I, OH, NH₂ and SH;
(5) C₁ to C₁₅ straight-chain or branched fluoroalkoxy, optionally substituted with at least one member selected from the group consisting of Cl, Br, I, OH, NH₂ and SH;
(6) C₁ to C₁₅ straight-chain or branched perfluoroalkyl; and
(7) C₁ to C₁₅ straight-chain or branched perfluoroalkoxy.

7. The process of claim 6 wherein said at least one acid catalyst is selected from the group consisting of:
(i) 1,1,2,2-tetrafluoroethanesulfonic acid;
(ii) 1,1,2,3,3,3-hexafluoropropanesulfonic acid;
(iii) 2-chloro-1,1,2-trifluoroethanesulfonic acid
(iv) 1,1,2-trifluoro-2-(perfluoroethoxy)ethanesulfonic acid;
(v) 1,1,2-trifluoro-2-(trifluoromethoxy)ethanesulfonic acid;
(vi) 1,1,2-trifluoro-2-(perfluoropropoxy)ethanesulfonic acid.

8. The process of claim 1 wherein the at least one acid catalyst is used at a concentration of from about 0.1% to about 20% by weight of the weight of the α-olefin(s) at the start of the reaction.

9. The process of claim 1 wherein the temperature is from about 50° C. to about 175° C.

10. The process of claim 1 wherein the reaction is carried out under an inert atmosphere at atmospheric pressure.

11. The process of claim 10 wherein the inert atmosphere is nitrogen, helium or argon.

12. The process of claim 1 wherein:
(i) Z⁺ is imidazolium or phosphonium and the length of the carbon chain of R¹, R², R³, R⁴, R⁵, R⁶ R⁷₁ R⁸, R⁹, or R¹⁰ is from four to twenty carbons;
(ii) A⁻ is selected from the group consisting of [CH₃OSO₃]⁻, [C₂H₅OSO₃]⁻, [CF₃SO₃]⁻, [HCF₂CF₂SO₃]⁻, [CF₃HFCCF₂SO₃]⁻, [HCClFCF₂SO₃]⁻, [(CF₃SO₂)₂N]⁻, [(CF₃CF₂SO₂)₂N]⁻, [CF₃OCFHCF₂SO₃]⁻, [CF₃CF₂OCFHCF₂SO₃]⁻, [CF₃CF₂CF₂OCFHCF₂SO₃]⁻, [CF₃CFHOCF₂CF₂SO₃]⁻, [CF₂HCF₂OCF₂CF₂SO₃]⁻, [CF₂ICF₂OCF₂CF₂SO₃]⁻, [CF₃CF₂OCF₂CF₂SO₃]⁻, and [(CF₂HCF₂SO₂)₂N]⁻, [(CF₃CFHCF₂SO₂)₂N]⁻;
(iii) said at least one acid catalyst is selected from the group consisting of 1,1,2,2-tetrafluoroethanesulfonic acid and 1,1,2,3,3,3-hexafluoropropanesulfonic acid; and
(vii) the temperature is from about 50° C. to about 175° C.

13. The process of claim 1 wherein the separated ionic liquid phase is reused to form the reaction mixture.
